(19) United States
(12) Patent Application Publication
(10) Pub. No.: US 2023/0355883 A1
(43) Pub. Date: Nov. 9, 2023

(54) MEDICAL DEVICE FOR VISUALLY DISPLAYING AN INJECTION PRESSURE OF A FLUID

(71) Applicant: B. Braun Melsungen AG, Melsungen (DE)

(72) Inventor: Nikolaus Berger-Roscher, Kassel (DE)

(21) Appl. No.: 18/022,956

(22) PCT Filed: Aug. 23, 2021
(86) PCT No.: PCT/EP2021/073301

§ 371 Date: Feb. 23, 2023

(30) Foreign Application Priority Data
Aug. 25, 2020 (DE) 10 2020 210 756.4

(51) Int. Cl. A61M 5/31; A61M 5/48; A61M 5/315

(52) U.S. Cl. CPC A61M 5/3134; A61M 5/31511; A61M 5/486; A61M 2205/0227; A61M 2205/3331

(57) ABSTRACT

A medical device and kit for visually displaying an injection pressure of a fluid, The device has a fluid path for the fluid and includes a piezochromic material which, under the action of a pressure of < 100 bar, displays a piezochromic color change that is perceptible to the human eye.

### CROSS-REFERENCE TO RELATED APPLICATIONS

This application is the U.S. National Stage Entry of International Application No. PCT/EP2021/073301, filed Aug. 23, 2021, and claims priority to German Application No. 10 2020 210 756.4, filed Aug. 25, 2020. The contents of International Application No. PCT/EP2021/073301 and German Application No. 10 2020 210 756.4 are incorporated by reference herein in their entireties.

### FIELD

The invention relates to a medical device for visually or optically displaying an injection pressure of a fluid, in particular a medical fluid.

### BACKGROUND

Many medical interventions are unperformable without appropriate anesthesia. A distinction is made between general anesthesia and local anesthesia. The most important part of local anesthesia is so-called regional anesthesia, in particular peripheral regional anesthesia.

Regional anesthesia involves administering an anesthetic close to the nerve that is responsible for transmission of stimuli from so-called nociceptors lying in the region of a planned medical intervention. A specific cannula is generally used for this purpose. If the cannula is guided too close to the nerves or into the nerves, temporary or even permanent nerve damage can occur. Nerve damage of this kind can also occur if the injection pressure of the anesthetic is too high. Therefore, both the positioning of the cannula and the injection pressure are crucial factors when performing (peripheral) regional anesthesia.

A device for visually displaying the pressure of a medical fluid, such as in particular an anesthetic, is known from WO 2003/101526 A1 for example. The device is inserted between a syringe and a cannula. The device has a flow channel which connects the syringe to the cannula and which communicates with a pressure chamber. The pressure chamber is delimited by a membrane. If the injection pressure rises in the flow channel and thus in the pressure chamber, the membrane is deflected and presses an indicating pin against the force of a spring, such that said indicating pin is pushed out of the housing of the device to a varying extent depending on the injection pressure prevailing in the flow channel, and as a result, the injection pressure can be visually monitored via color ring coding on the indicating pin.

A device for limiting the injection pressure of a medical instrument for introducing a fluid is known from WO 2017/071833 A1. In the case of this device, the injection pressure is limited by a valve. When a threshold pressure is reached, further supply of fluid is interrupted. The counterforce required to close the valve is likewise brought about by a spring.

Furthermore, WO 2018/170231 A1 discloses an injection syringe having a piezochromic pressure indicator which indicates the pressure of a fluid discharged from the injection syringe via a color change.

Comparatively complex devices for measuring a pressure of medical fluids are known from the documents US 2013/0165904 A1 and US 2018/0064870 A1.

Conventional devices for visually displaying an injection pressure of fluids commonly have the disadvantage of either having a highly complex structure or having color pressure indicators which either only respond at relatively high pressures and/or have a distinctly delayed pressure response behavior, thereby greatly limiting their use for medical applications, especially regional anesthetic applications.

### SUMMARY

It is an object of the invention to provide a device for visually or optically displaying an injection pressure of a fluid, in particular a medical fluid such as an anesthetic, which device at least partially or even completely avoids the disadvantages described in the introduction in connection with conventional devices.

The invention relates to a medical device for visually or optically displaying an injection pressure, in particular an injection threshold pressure, of a fluid, preferably a medical fluid, in particular an anesthetic or an anesthetic-containing liquid.

The medical device comprises a fluid path for the fluid, i.e., a fluid path through which the fluid is conveyed, and a piezochromic material.

The invention is particularly distinguished by the fact that the piezochromic material exhibits a piezochromic color change perceptible to the human eye when exposed to a pressure of < (pronounced: “less than”) 100 bar.

The expression “injection pressure” is to be understood in the context of the present invention to mean a pressure, preferably a static pressure, that is exerted by a fluid on body cells, in particular nerve cells, and/or body tissue, in particular nerve tissue, and/or other body structures when said fluid is injected into the body of a patient.

The expression “pressure” is to be understood in the context of the present invention to mean an absolute pressure, i.e., a pressure that is based on a zero pressure prevailing in an airless space or in a vacuum, in particular the universe.

The expression “injection threshold pressure” is to be understood in the context of the present invention to mean a pressure threshold value, preferably a threshold value of a static pressure, values above which are to be avoided as far as possible from preferably a medical perspective, in particular from the perspective of cell damage, in particular nerve cell damage, and/or body tissue damage, in particular nerve tissue damage.

The expression “fluid” is to be understood in the context of the present invention to mean a liquid, preferably a medical liquid, in particular an anesthetic or an anesthetic-containing liquid.

The expression “piezochromic material” is to be understood in the context of the present invention to mean a material that changes color when exposed to pressure.

The invention is based on the surprising finding that a piezochromic material that changes color when exposed to a pressure of < 100 bar is suitable for visually or optically displaying the event of one or more injection threshold pressures of a fluid, preferably a medical fluid, such as in particular an anesthetic, having been reached or exceeded. This is especially advantageous when performing peripheral regional anesthesia. With this kind of anesthesia, the anesthesiologist brings the tip of the cannula as close as possible to the nerve in question, for example by means of ultrasound guidance. The anesthesiologist then checks the correct position of the tip of the cannula by carefully increasing the force on the plunger of the syringe. If the cannula tip is directly on the nerve or even in the nerve, the opening injection pressure of the anesthetic generally rises to a value of > (pronounced: “greater than”) 15 psi (1.034 bar). The injection pressure is static or substantially static, since the fluid flow of the anesthetic is negligible. The static liquid pressure, which is the same throughout the fluid path, can, now, be shown via the device according to the invention or the piezochromic material. This gives the anesthesiologist the opportunity to correct the position of the cannula tip if necessary. Consequently, the risk of nerve damage when performing regional anesthesia can be significantly reduced.

Advantageously, depending on the kind of piezochromic material and the chemical composition thereof, it is possible for threshold pressures, i.e., pressure threshold values, pressures above which may be unsafe from a medical perspective for example, to be visually displayed highly accurately, i.e., without a large range of fluctuations, and in particular reliably and reproducibly. For example, with the aid of the device according to the invention, it is possible to visualize injection threshold pressures, pressures above which lead to damage to body cells, in particular nerve cells, and/or body tissues, in particular nerve tissue.

A further advantage is that the medical device according to the invention has a significantly faster pressure response behavior than pressure indicators known from the prior art owing to the piezochromic material. The pressure sensitivity of the piezochromic material, which is improved compared to pressure indicators of the type in question, can additionally optimize the visualization and observance of especially medically relevant injection threshold pressures.

A further advantage of the invention is that the piezochromic material can also be used to measure pressures which are of particular relevance for medical applications, in particular in the field of regional anesthesia, preferably peripheral regional anesthesia.

Furthermore, it is advantageous that the medical device according to the invention can have a significantly simpler structure compared to conventional devices. In particular, the medical device according to the invention can comprise fewer components than devices known from the prior art. Especially preferably, the device according to the invention, apart from a possible syringe plunger, can comprise no movable components, in particular no mechanically movable components, and/or no electronic components. As a result, relatively inexpensive manufacture of the medical device according to the invention is realizable. The medical device according to the invention can therefore preferably be designed as a disposable product, i.e., as a product intended for single use. Alternatively, the medical device according to the present invention can be designed as a reusable product, i.e., as a product intended for multiple use.

Lastly, it is advantageous that the medical device according to the invention can be provided with any form of connectors, for example Luer connectors, connectors available under the registered trademark NRFIT®, and the like.

In one embodiment of the invention, the piezochromic material exhibits a piezochromic color change perceptible to the human eye when exposed to a pressure of < (pronounced: “less than”) 50 bar, in particular (pronounced: “less than”) 10 bar, preferably (pronounced: “less than”) 5 bar, particularly preferably from 1 bar to 3 bar. It is especially the pressure sensitivity of the piezochromic material as disclosed in this paragraph that advantageously contributes to preventing an injection threshold pressure critical from a medical perspective from being exceeded and to thus preventing damage to body cells and/or body tissue, in particular nerve cells and/or nerve tissue.

In a further embodiment of the invention, the piezochromic material exhibits the piezochromic color change perceptible to the human eye within a period of < (pronounced: “less than”) 5 s, in particular within a period of < (pronounced: “less than”) 1 s. In particular, the piezochromic material can exhibit the piezochromic color change perceptible to the human eye within a period of 0 s, in particular > (pronounced: “greater than”) 0 s, to 2 s, preferably within a period of 0 s, in particular > (pronounced: “greater than”) 0 s, to 1 s. As a result, a virtually immediate color change of the piezochromic material is achievable upon pressure exposure. This advantageously likewise contributes to avoiding pressures above an injection threshold pressure which is unsafe from a medical perspective.

In principle, the piezochromic material can exhibit a reversible or irreversible piezochromic color change. For example, preference may be given to a reversible color change if the medical device is to be used multiple times or the fluid is to be injected at intervals, i.e., portions of the fluid are to be injected one after the other. Alternatively, preference may be given to an irreversible color change of the piezochromic material. For example, such a color change of the piezochromic material may be advantageous for proof that specific injection pressures were not exceeded in a medical application.

Furthermore, the piezochromic material, when exposed to the pressure, can exhibit a so-called bathochromic effect, i.e., a red shift or deepening of color, i.e., a shift in its absorption spectrum to the longer-wave, lower-energy region of the electromagnetic spectrum. Alternatively, the piezochromic material, when exposed to the pressure, can exhibit a so-called hypsochromic effect, i.e., a blue shift, i.e., the shift in its absorption spectrum to the shorter-wave, higher-energy region of the electromagnetic spectrum.

Furthermore, the piezochromic material is preferably a sterilization-resistant material, in particular a material which is resistant to gas sterilization, preferably ethylene oxide sterilization.

Preferably, the piezochromic material comprises a polymer and/or copolymer. Alternatively, the piezochromic material can be a polymer and/or copolymer.

The expression “copolymer” is to be understood in the context of the present invention to mean a polymer which has at least two different monomer units.

In a further embodiment of the invention, the piezochromic material comprises a matrix composed of at least one polymer and/or copolymer, at least one liquid crystal and at least one optically active substance. The at least one optically active substance is preferably distributed in the matrix.

Furthermore, the at least one optically active substance is preferably a chiral substance or chirally optically active substance.

In principle, all known liquid crystals can be used as the liquid crystal.

In a further embodiment of the invention, the at least one liquid crystal is selected from the group consisting of N-(p-ethoxybenzylidene)-p-n-butylaniline, N-(p-methoxybenzylidene)-p-n-butylaniline, 4-alkylphenyl 4-n-alkylbenzoate, cholesteryl benzoate, cholesterol, tolan, alkanoic acids, stilbene, azobenzene, 4-phenylcinnamic acid, p-terphenyl, 1,2-bisbenzoethylene and mixtures of at least two of the aforementioned liquid crystals.

In a further embodiment of the invention, the at least one optically active substance is selected from the group consisting of 4-(4-hexyloxybenzoyloxy)benzoate, 2-octyl 4-[[4-(hexoyl)benzoyl]oxy]benzoate, cholesteryl derivatives and mixtures of at least two of the aforementioned optically active substances.

In a further embodiment of the invention, the polymer and/or copolymer of the piezochromic material is/are a polyacrylate, polymethacrylate, copolyacrylate, copolymethacrylate or a mixture of at least two of the aforementioned polymers and/or copolymers. The copolyacrylates and/or copolymethacrylates have preferably been synthesized from at least one monofunctional monomer and/or at least one multifunctional crosslinker.

In a further embodiment of the invention, the monofunctional monomer is selected from the group consisting of benzyl acrylate, benzyl methacrylate, 2-ethylhexyl acrylate, 2-ethylhexyl methacrylate, methoxyethyl acrylate, methoxyethyl methacrylate, octadecyl acrylate, octadecyl methacrylate and mixtures of at least two of the aforementioned monomers.

In a further embodiment, the crosslinker is selected from the group consisting of 1,4-butanediol diacrylate, 1,4-butanediol dimethacrylate, polyethylene glycol diacrylate, polyethylene glycol dimethacrylate, 1,10-decanediol diacrylate, 1,10-decanediol dimethacrylate, bisphenol A glycerolate (1 glycerol/phenol) diacrylate and mixtures of at least two of the aforementioned crosslinkers.

Preferably, the crosslinker and the monofunctional monomer have a weight ratio (crosslinker to monofunctional monomer) of between 0.01 and 0.8, preferably between 0.1 and 0.6, particularly preferably between 0.2 and 0.4.

Furthermore, the at least one liquid crystal preferably has a proportion of up to 90% by weight, in particular between 15% by weight and 60% by weight, preferably between 25% by weight and 32% by weight, based on the total weight of the piezochromic material or the matrix of the piezochromic material.

Furthermore, the at least one optically active substance preferably has a proportion of up to 10% by weight, in particular between 0.1% by weight and 2.5% by weight, preferably between 0.15% by weight and 0.55% by weight, based on the total weight of the piezochromic material or the matrix of the piezochromic material.

Furthermore, the piezochromic material is preferably in the form of an elastic and highly ordered system. Preferably, the system has a helical structure.

Regarding further features and advantages of the piezochromic material, reference is made to WO 2011/012315 A1, the disclosure content of which with regard to the piezochromic material described there is incorporated in the present description by express reference.

In a further embodiment of the invention, the piezochromic material is contained in a wall of a housing of the medical device that surrounds the fluid path, i.e., in a housing wall surrounding the fluid path. The piezochromic material can be contained only in a single wall section of the housing wall of the medical device. Alternatively, preference may be given to the piezochromic material being contained in a plurality of wall sections of the housing wall of the medical device. The wall sections can be arranged in the longitudinal direction and/or in the transverse or circumferential direction of the medical device. In the case of a medical device having a cylindrical, in particular circular-cylindrical, housing, the wall sections can also be arranged radially. In particular, the wall sections can differ from one another with regard to the piezochromic material. As a result, different injection pressures, in particular injection threshold pressures, can be shown visually or optically with particular advantage.

In a further embodiment of the invention, the housing wall of the medical device, preferably in the thickness direction of the housing wall, comprises at least one multilayer, in particular trilayer, wall section, i.e., only one multilayer, in particular trilayer, wall section or multiple multilayer, in particular trilayer, wall sections. Preferably, an intermediate layer of the at least one multilayer wall section comprises the piezochromic material or consists of the piezochromic material. In particular, the intermediate layer, in particular in the longitudinal or circumferential direction of the housing of the medical device, can be composed of multiple intermediate layer sections arranged next to one another, preferably directly next to one another. Preferably, the intermediate layer sections differ from one another with regard to the piezochromic material. As a result, different injection pressures can be shown optically with particular advantage. Preferably, the intermediate layer has a layer thickness of 0 mm, in particular > (pronounced: “greater than”) 0 mm, to 1 mm, in particular 0 mm, in particular > (pronounced: “greater than”) 0 mm, to 0.6 mm, preferably 0 mm, in particular > (pronounced: “greater than”) 0 mm, to 0.2 mm.

In a further embodiment of the invention, the at least one multilayer, in particular trilayer, wall section also comprises an inner layer which covers the intermediate layer on the inner face, i.e., on a side facing the fluid path, preferably directly and in particular completely. Particularly preferably, the inner layer is in direct contact with the fluid path. The inner layer preferably comprises a biocompatible, i.e., medically acceptable, material or preferably consists of such a material. As a result, protection of the piezochromic material from direct contact with the fluid and thus avoidance of any impairment of the piezochromic material by the fluid or constituents thereof is advantageously achievable. Preferably, the inner layer has a layer thickness of 0 mm, in particular > (pronounced: “greater than”) 0 mm, to 1 mm, in particular 0 mm, in particular > (pronounced: “greater than”) 0 mm, to 0.3 mm, preferably 0 mm, in particular > (pronounced: “greater than”) 0 mm, to 0.1 mm. Such a layer thickness advantageously ensures that the pressure exerted by the fluid can be virtually directly transmitted onto the piezochromic material and that the color change of the piezochromic material can thus be brought about reliably and reproducibly.

In a further embodiment of the invention, the biocompatible material is selected from the group consisting of polyethylene terephthalate (PET), polyetheretherketone (PEEK), polypropylene (PP), polyphenylsulfone (PPSU), polyoxymethylene (POM) and mixtures of at least two of the aforementioned biocompatible materials.

In a further embodiment of the invention, the at least one multilayer, in particular trilayer, wall section also comprises an outer layer which covers the intermediate layer on the outer face, i.e., on a side facing away from the fluid path, preferably directly and in particular completely. Particularly preferably, the outer layer forms part of the exterior of the medical device, in particular the housing of the medical device. Preferably, the outer layer comprises a transparent, i.e., light-transmissive, material. Alternatively, the outer layer can preferably consist of such a material. As a result, a visual or optical display of the color change of the piezochromic material is advantageously achievable. The outer layer can be, in particular, designed as a screen or display.

The expression “transparent material” or “light-transmissive material” is to be understood in the context of the present invention to mean a material which is transmissive to light having a wavelength of 380 nm to 780 nm.

In a further embodiment of the invention, the transparent material is selected from the group consisting of polycarbonate (PC), polymethyl methacrylate (PMMA), polyurethane (PU) and mixtures of at least two of the aforementioned transparent materials.

Particularly preferably, the at least one multilayer, in particular trilayer, wall section of the housing wall of the medical device is made up, from the inside to the outside, of an inner layer, intermediate layer and outer layer, as described in the previous paragraphs.

As already mentioned, the piezochromic material is preferably contained in a wall of a housing of the medical device. The housing can, in principle, have any conceivable shape suitable for forming or defining a fluid path through which the fluid can be conveyed. For example, the housing can have a prismatic shape, in particular a cubic or cuboid shape, or a cylindrical shape, in particular a circular-cylindrical shape.

Preferably, the medical device, in particular the housing of the medical device, comprises, preferably at a distal end of the medical device, in particular the housing of the medical device, a first connector mechanism for connecting the medical device, in particular the housing of the medical device, to an injection tube. The expression “distal end” is to be understood here to mean the end of the medical device, in particular the housing of the medical device, that is remote from the center of the body of a user, in particular an anesthesiologist.

Further preferably, the medical device, in particular the housing of the medical device, comprises, preferably at a proximal end of the medical device, in particular the housing of the medical device, a second connector mechanism for connecting the medical device, in particular the housing of the medical device, to a syringe body. The expression “proximal end” is to be understood here to mean the end of the medical device, in particular the housing of the medical device, that is towards the center of the body of a user, in particular an anesthesiologist.

The connector mechanisms described in the previous paragraphs can be, for example, in the form of Luer-Lock connectors, in particular in the form of male Luer-Lock connectors, female Luer-Lock connectors, male Luer-Lock connector and female Luer-Lock connector, or connectors available under the registered trademark NRFIT®.

In a further embodiment of the invention, the housing wall is a wall of a syringe body of the medical device.

In a further embodiment of the invention, the housing wall is a lateral wall of a syringe body of the medical device. The expression “lateral wall” is be understood in this connection to mean a wall forming the lateral surface of the syringe body.

In a further embodiment of the invention, the housing wall is an end wall of a syringe body of the medical device. The expression “end wall” is to be understood in this connection to mean a wall of the syringe body that is broken through or interrupted by an outlet opening which opens into a nozzle, preferably a conical or cylindrical nozzle, of the syringe body.

Especially preferably, the multilayer, in particular trilayer, wall section of the housing wall can be an end wall of a syringe body of the medical device.

In a further embodiment of the invention, the housing wall is a wall of a nozzle, in particular a conical or cylindrical nozzle, of a syringe body of the medical device.

In a further embodiment of the invention, the nozzle, in particular the conical or cylindrical nozzle, of the syringe body comprises, in particular orthogonally to the longitudinal direction of the nozzle, a branch or bifurcation having a closed end. Preferably, the piezochromic material is contained in the closed end of the branch or bifurcation. Especially preferably, the closed end of the branch or bifurcation comprises the at least one wall section composed of multiple layers, in particular three layers, in the thickness direction of the housing wall that has already been mentioned multiple times, or is formed therefrom.

Preferably, the syringe body mentioned in the previous paragraphs is a syringe barrel in each case. The syringe body, in particular the syringe barrel, can have an elliptical or circular cross section. Preferably, the syringe body, in particular the syringe barrel, is circular-cylindrical.

In a further embodiment of the invention, the piezochromic material is contained in a stopper of a syringe plunger of the medical device. In particular, the piezochromic material can be layered in the stopper of the syringe plunger. Further preferably, the piezochromic material is coated with a biocompatible material. Preferably, the biocompatible material forms an outer layer, i.e., an outermost layer, of the stopper. Regarding suitable biocompatible materials, full reference is made to the biocompatible materials already described in the foregoing description.

Preferably, the medical device in the embodiments of the invention described in the previous paragraphs is designed as an injection device for injecting a fluid, preferably as a syringe.

Furthermore, the medical device is preferably sterilization-resistant, in particular resistant to gas sterilization, preferably ethylene oxide sterilization.

The invention further relates to a medical kit for visually or optically displaying an injection pressure, in particular an injection threshold pressure, of a fluid, preferably a medical fluid, such as in particular an anesthetic or an anesthetic-containing liquid.

The kit spatially separately comprises the following:
a medical device according to the invention and
at least one further component selected from the group consisting of cannula, in particular for peripheral nerve blocks, injection tube, injection device, in particular syringe, connectors such as Luer connector and/or connectors available under the registered trademark NRFIT®, and combinations of at least two of the aforementioned further components.

Regarding further features and advantages of the kit, in particular the medical device according to the invention, full reference is made to the foregoing description.

Further features and advantages of the invention will become apparent from the claims and from the following description of preferred embodiments with reference to figures and to examples. Here, features of the invention can each be realized alone or in combination with one another. The embodiments described hereinbelow serve for further elucidation of the invention, but without restricting the invention thereto.

### BRIEF DESCRIPTION OF THE DRAWINGS

The following is shown schematically in the figures:

FIG. 1: one embodiment of a medical device according to the invention,

FIG. 2: a cross-sectional representation of a further embodiment of a medical device according to the invention,

FIG. 3: a plan view of the embodiment of a medical device according to the invention depicted in FIG. 2,

FIG. 4: a detailed view of a housing wall of a further embodiment of a medical device according to the invention,

FIG. 5: a plan view of a housing wall of a further embodiment of a medical device according to the invention,

FIG. 6: a further embodiment of a medical device according to the invention,

FIG. 7: a further embodiment of a medical device according to the invention,

FIG. 8: a further embodiment of a medical device according to the invention, and

FIG. 9 a further embodiment of a medical device according to the invention.

### DETAILED DESCRIPTION

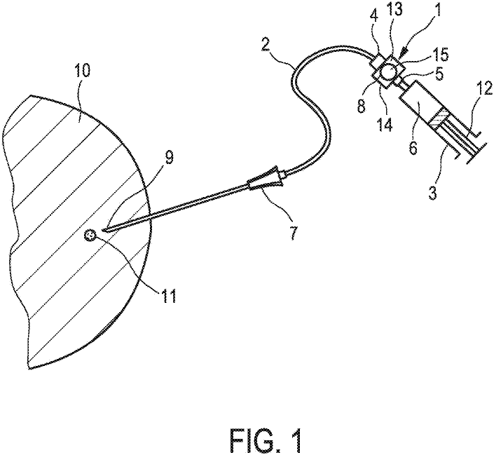

FIG. 1 shows schematically one embodiment of a medical device 1 according to the invention for visually or optically displaying an injection pressure, in particular an injection threshold pressure, of an anesthetic when performing peripheral regional anesthesia.

The medical device 1 is connected between an injection tube 2 and a syringe 3. To this end, the medical device advantageously comprises a connector mechanism 4 for connecting the medical device 1 to the injection tube 2 and a further connector mechanism 5 for connecting the medical device 1 to the syringe 3. The connector mechanisms 4 and 5 can, for example, each be in the form of Luer-Lock connectors.

The syringe 3 has a cavity (lumen) 6 which has been filled with an anesthetic. A cannula 7 is attached to a distal end of the injection tube 2, i.e., to an end remote from the center of the body of a user, in this case an anesthesiologist.

The medical device 1 comprises a fluid path 8 for the anesthetic and a piezochromic material. The piezochromic material preferably exhibits a piezochromic color change perceptible to the human eye when exposed to a pressure of < 10 bar, preferably < 5 bar, particularly preferably from 1 bar to 3 bar. The piezochromic material can be, in particular, a piezochromic plastic or a piezochromic composition. Preferably, the piezochromic material comprises a matrix composed of at least one polymer and/or copolymer, at least one liquid crystal and at least one optically active substance, in particular a chirally optically active substance. The optically active substance is preferably distributed in the matrix.

The aforementioned polymer can be, for example, a crosslinked polymethacrylate. The aforementioned copolymer can be, for example, a crosslinked copolymethacrylate. The at least one liquid crystal can be, for example, a liquid-crystal mixture of four different alkylcyanobiphenyl derivatives and one alkoxycyanobiphenyl derivative. The optically active substance can be, for example, the substance S-811, i.e., 4-(4-hexyloxybenzoyloxy)benzoate, cholesteryl derivatives and/or mixtures thereof.

To perform the peripheral regional anesthesia, the tip 9 of the cannula 7 is brought as close as possible to a nerve 11 located in a peripheral tissue region 10, for example under ultrasound guidance. The nerve 11 is depicted in cross section in FIG. 1. In addition, the anesthesiologist checks the correct position of the cannula tip 9 by carefully increasing the force on a plunger 12 of the syringe 3. If the tip 9 of the cannula 7 is directly on the nerve 11 or even in the nerve 11, the opening injection pressure, which is in principle static, rises to a defined value, generally to a value greater than 1.034 bar (> 15 psi). The static pressure of the anesthetic, which is the same throughout the fluid path 8 of the medical device 1, can, now, be shown optically via the piezochromic material of the medical device 1. This is achieved by the fluid path 8, which runs through the medical device 1, being guided past a housing wall 13, i.e., past a wall 13 of a housing 14 of the medical device 1, said housing wall 13 comprising in the thickness direction thereof at least one wall section 15 which is composed of multiple layers, in particular three layers, and contains the piezochromic material. Preferably, the wall section 15 comprises an intermediate layer which has been coated with an inner layer on a side facing the fluid path 8 and with an outer layer on a side facing away from the fluid path 8. Preferably, the intermediate layer comprises the piezochromic material or consists of the piezochromic material. The inner layer preferably comprises a biocompatible material, preferably for protection of the piezochromic material from the fluid, or consists of such a material. The biocompatible material can be, for example, polyethylene terephthalate, polyetheretherketone, polypropylene, polyphenylsulfone or polyoxymethylene. The outer layer preferably comprises a transparent material or consists of such a material. The transparent material can be, for example, polycarbonate, polymethyl methacrylate or polyurethane. Conveniently, the inner layer is in direct contact with the fluid path 8 of the medical device 1, whereas the outer layer conveniently forms part of the exterior of the housing 14 of the medical device 1. The outer layer can be, in particular, designed as a screen or display.

The piezochromic material changes its color when the injection pressure is increased to a defined value dependent on the composition of the piezochromic material. The color change can occur reversibly or irreversibly. Advantageously, the threshold pressure of the injection pressure can be adjusted very accurately and in particular reproducibly via the composition of the piezochromic material. In this way, pressures which reach or exceed a defined pressure can be seen optically. Owing to the abovementioned transparent outer layer, the color change of the piezochromic material is outwardly visible. As a result, the anesthesiologist will detect that he has positioned the cannula tip 9 either too close to the nerve to be blocked or even in said nerve, and can appropriately correct the position of the cannula tip 9. As a result, damage to the nerve 11 can be avoided with particular advantage.

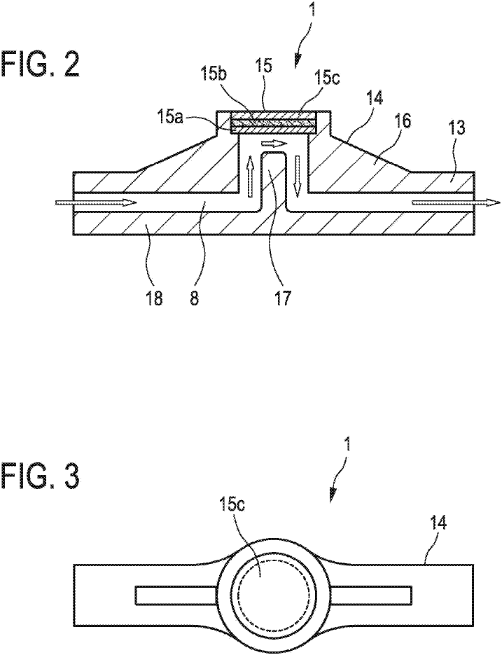

FIG. 2 shows schematically a cross-sectional view of a further embodiment of a medical device 1 according to the invention for visually or optically displaying an injection pressure, in particular an injection threshold pressure, of a fluid, for example an anesthetic or an anesthetic-containing liquid.

The medical device 1 comprises a fluid path 8 which is surrounded by a housing wall 13, i.e., a wall 13 of a housing 14 of the medical device 1. The wall 11 comprises a wall section 15. The wall section 15 has a trilayer structure composed of an inner layer 15a, an intermediate layer 15b and an outer layer 15c. The intermediate layer 15b comprises a piezochromic material or consists of such a material. The inner layer 15a comprises a biocompatible material for protection of the piezochromic material from direct contact with the fluid or consists of such a material. The outer layer 15c comprises a transparent material or consists of such a material. Regarding further features and advantages of the piezochromic material, biocompatible material and transparent material, reference is made to the foregoing description, in particular to the features and advantages mentioned in the figure description for FIG. 1.

The inner layer 15a covers the intermediate layer 15b, preferably directly, on a side facing the fluid path 8. The inner layer 15a is preferably in direct contact with the fluid path 8. In contrast, the outer layer 15c covers the intermediate layer 15b, preferably directly, on a side facing away from the fluid path 8. Preferably, the outer layer 15c forms part of the exterior of the housing 14 of the medical device 1.

The housing 14 of the medical device 1 can further comprise a reinforcement, in particular a reinforcing rib, 16, in particular in the region of the wall section 15. Furthermore, the medical device 1 can comprise a web 17 formed on the inside toward the wall section 15. Preferably, the web 17 is part of a part 18 of the housing 14 that is arranged opposite the wall section 15. The web 17 can advantageously prevent the formation of air bubbles when injecting the fluid and guide the fluid flow directly past the wall section 15 and thus past the piezochromic material present therein. Furthermore, a reduction in the dead space volume is advantageously realizable as a result.

For further functionality of the medical device 1 shown schematically in FIG. 2, full reference is made to the foregoing description, in particular to the figure description for FIG. 1. The discussion there in this respect applies correspondingly.

FIG. 3 shows schematically a plan view of the embodiment of a medical device 1 according to the invention depicted in FIG. 2.

A color change of the piezochromic material, and thus pressures which reach or exceed an injection threshold pressure when injecting the fluid, is optically detectable via the transparent outer layer 15c located on the exterior of the housing 14. If necessary, an anesthesiologist can correct the position of a cannula tip, especially when performing a peripheral regional anesthetic procedure.

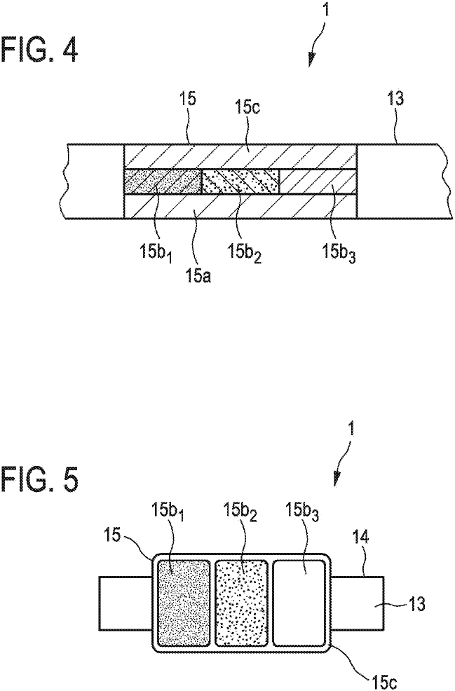

FIG. 4 shows schematically the detailed view of a housing wall 13 of a further embodiment of a medical device according to the invention for visually or optically displaying an injection pressure, in particular an injection threshold pressure, of a fluid, for example an anesthetic or an anesthetic-containing liquid.

The housing wall 13 comprises a wall section 15. It has, in the thickness direction of the housing wall 13, a trilayer structure composed of an inner layer 15a, an intermediate layer 15b and an outer layer 15c. The intermediate layer 15b consists of multiple intermediate layer sections which are arranged next to one another, preferably directly next to one another, in the longitudinal direction of the housing wall. Preferably, the intermediate layer sections each comprise a different piezochromic material or each consist of a different piezochromic material. As a result, different injection threshold pressures can be advantageously visualized. For example, the intermediate layer 15b, as depicted in FIG. 4, can consist of three such intermediate layer sections 15b₁, 15b₂ and 15b₃. For example, the piezochromic materials for the intermediate layer sections 15bi, 15b₂ and 15b₃ can be chosen such that the piezochromic material of the intermediate layer section 15b₁ exhibits a color change when exposed to a pressure ≥ 0.69 bar (≥ 10 psi), the piezochromic material of the intermediate layer section 15b₂ exhibits a color change when exposed to a pressure ≥ 1.03 bar (≥ 15 psi) and the piezochromic material of the intermediate layer section 15b₃ exhibits a color change when exposed to a pressure ≥ 1.38 bar (≥ 20 psi). Regarding further suitable piezochromic materials, reference is made to the piezochromic materials already mentioned in the foregoing description, in particular in the figure description for FIG. 1.

On a side facing a fluid path, the intermediate layer 15b is covered, preferably directly and completely, by the inner layer 15a. Preferably, the inner layer 15a is in direct contact with the fluid path. The inner layer 15a comprises a biocompatible material for protection of the piezochromic materials of the intermediate layer 15b from direct contact with a fluid which is conveyed through the fluid path, or consists of such a material. Regarding suitable biocompatible materials, reference is made to the biocompatible materials already mentioned in the foregoing description, in particular in the figure description for FIG. 1.

The outer layer 15c covers, preferably directly and completely, the intermediate layer 15b preferably on a side facing away from a fluid path. Preferably, the outer layer 15c forms part of the exterior of the housing 14 of the medical device 1. The inner layer 15c comprises a transparent material or consists of such a material. As a result, a color change of the piezochromic materials will be optically detectable for a user, in particular an anesthesiologist. The outer layer can be, in particular, designed as a screen or display.

Regarding suitable transparent materials, reference is made to the transparent materials already mentioned in the foregoing description, in particular in the figure description for FIG. 1.

FIG. 5 shows schematically a plan view of a housing wall of a further embodiment of a medical device 1 according to the invention.

The medical device 1 comprises a housing 14 having a housing wall 13. The housing wall 13 comprises a wall section 15 which is widened in the thickness direction of the housing wall compared to the rest of the housing wall. The wall section 15 has the structure described in the figure description for FIG. 4. Via the outer layer 15c, which can be in particular designed as a screen or display, it is possible to visualize the color changes caused by the piezochromic materials of the intermediate layer sections 15b₁, 15b₂ and 15b₃ when exposed to different pressures.

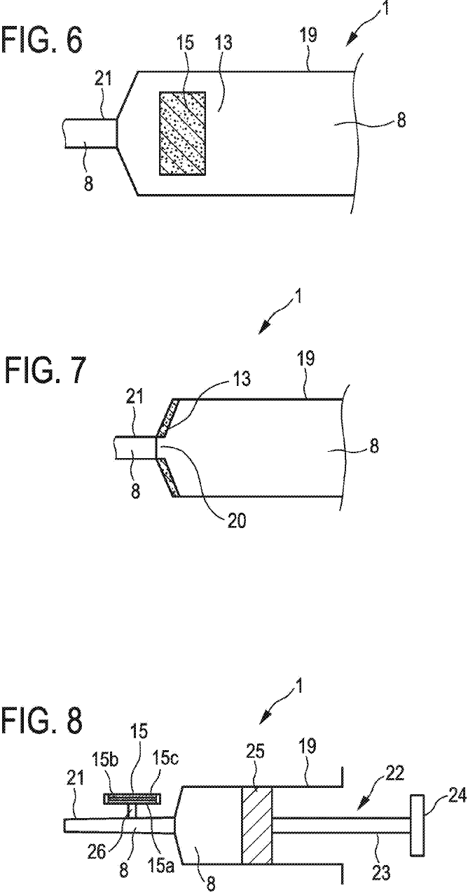

FIG. 6 shows schematically a further embodiment of a medical device 1 according to the invention for visually or optically displaying an injection pressure, in particular an injection threshold pressure, of a fluid, for example an anesthetic or an anesthetic-containing liquid.

The medical device 1 is designed as a syringe having a syringe body 19 having a preferably conical or cylindrical outlet nozzle 21. The syringe body 19 is preferably cylindrical, in particular circular-cylindrical. The medical device 1 comprises a fluid path 8 which is formed both by the cavity of the syringe body 19 and by the cavity of the outlet nozzle 21.

Furthermore, the syringe body 19 comprises a lateral wall 13 having a wall section 15. The wall section 15 has, in the thickness direction of the lateral wall, a trilayer structure composed of an inner layer, intermediate layer and outer layer. The inner layer covers the intermediate layer on a side facing the fluid path 8, whereas the outer layer covers the intermediate layer on a side facing away from the fluid path 8. Preferably, the inner layer is in direct contact with the fluid path 8. Preferably, the outer layer forms part of the exterior of the syringe body 19. The intermediate layer comprises a piezochromic material or consists of a piezochromic material. The inner layer comprises a biocompatible material for protection of the piezochromic material from direct contact with the fluid or consists of such a biocompatible material. The outer layer comprises a transparent material or consists of a transparent material. Regarding suitable piezochromic materials, biocompatible materials and transparent materials and regarding the functionality of the medical device 1, reference is made to the foregoing description, in particular to the figure description for FIG. 1.

FIG. 7 shows schematically a further embodiment of a medical device 1 according to the invention for visually or optically displaying an injection pressure, in particular an injection threshold pressure, of a fluid, for example an anesthetic or an anesthetic-containing liquid.

The medical device 1 is (likewise) designed as a syringe having a syringe body 19. The syringe body 19 is preferably cylindrical, in particular circular-cylindrical. The medical device 1 comprises a fluid path 8 which is formed both by the cavity of the syringe body 19 and by the cavity of the outlet nozzle 21.

Furthermore, the syringe body 19 comprises an end wall 13 having a trilayer structure composed of an inner layer, intermediate layer and outer layer. The end wall surrounds an outlet opening 20 which opens into a conical or cylindrical outlet nozzle 21 of the syringe body 19.

Regarding further features and advantages and regarding the further functionality of the medical device shown in FIG. 7, in particular regarding the aforementioned intermediate layer, inner layer and outer layer, full reference is made to the foregoing description, in particular to the figure descriptions for FIGS. 1 and 6.

FIG. 8 shows a further embodiment of a medical device 1 according to the invention for visually or optically displaying an injection pressure, in particular an injection threshold pressure, of a fluid, for example an anesthetic or an anesthetic-containing liquid.

The medical device 1 is (likewise) designed as a syringe and comprises a syringe body 19 having a preferably conical or cylindrical outlet nozzle 21 and a syringe plunger 22. The syringe body 19 is preferably cylindrical, in particular circular-cylindrical. The syringe plunger 22 comprises a rod 23, a head 24 and a stopper 25. The medical device 1 comprises a fluid path 8 which is formed both by the cavity of the syringe body 19 and by the cavity of the outlet nozzle 21.

The outlet nozzle 21 has a branch or bifurcation 26 having a closed end 15. The closed end 15 thus forms a branched-off or bifurcated wall section of the syringe body 19.

Preferably, the closed end or the branched-off or bifurcated wall section 15 has a trilayer structure composed of an inner layer 15a, intermediate layer 15b and outer layer 15c.

Regarding further features and advantages and regarding the further functionality of the medical device shown in FIG. 8, in particular regarding the aforementioned intermediate layer, inner layer and outer layer, full reference is made to the foregoing description, in particular to the figure descriptions for FIGS. 1 and 6.

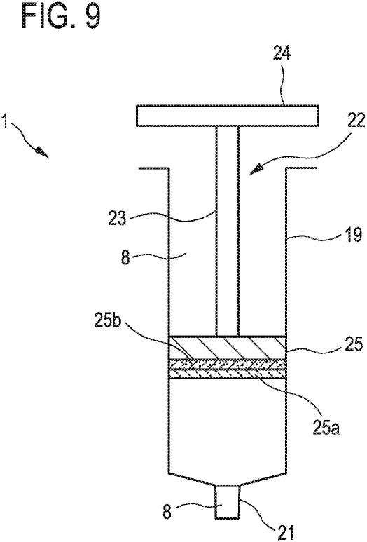

FIG. 9 shows a further embodiment of a medical device 1 according to the invention for visually or optically displaying an injection pressure, in particular an injection threshold pressure, of a fluid, for example an anesthetic or an anesthetic-containing liquid.

The medical device 1 is (likewise) designed as a syringe and has a syringe body 19 having a preferably conical or cylindrical outlet nozzle 21 and a syringe plunger 22 having a rod 23, a head 24 and a stopper 25. The medical device 1 comprises a fluid path 8 which is formed both by the cavity of the syringe body 19 and by the cavity of the outlet nozzle 21.

The stopper 25 of the syringe plunger 22 comprises a layer 25b which is covered by a layer 25a, said layer 25a forming part of the exterior of the stopper 25. The layer 25b comprises a piezochromic material or consists of such a material. The layer 25a comprises a transparent material or consists of a transparent material. In this way, a color change of the piezochromic material can be visualized for a user, in particular a physician.

Regarding further features and advantages and regarding the further functionality of the medical device shown in FIG. 8, in particular regarding the aforementioned piezochromic material and the aforementioned transparent material, full reference is made to the foregoing description, in particular to the figure descriptions for FIGS. 1 and 6.

### EXAMPLE SECTION

### 1. Preparation of a Piezochromic Material According to the Present Invention (Including an Appropriately Assembled Film)

### 1.1 Preparation of a Mixture of a Liquid Crystal and a Chirally Optically Active Substance

The optically active substance S-811 was admixed at a proportion by weight of 22% to a liquid crystal mixture E5 (mixture consisting of four alkylcyanobiphenyl derivative components and one alkoxycyanobiphenyl derivative component). The mixture was heated above the clearing point of the E5 component to 58° C. and then cooled back to room temperature.

### 1.2 Preparation of a Mixture of a Monofunctional Methacrylate Monomer and a Crosslinker

34% by weight of DDA was doped into the monomer benzyl methacrylate. Thereafter, the mixture was heated to 60° C. for 25 min.

The mixtures prepared according to 1.1 and 1.2 were mixed in a ratio of 1:2.4 at a temperature between 50° C. and 55° C. Finally, 0.2% by weight of Genocure LTM polymerization initiator was doped in. The piezochromic material was assembled with a layer thickness of 25 µm between two polypropylene films, which were each 15 µm thick and which were polyamide-coated (less than 1 µm), and polymerized under UV light for 15 min. This yielded a red-reflecting film which was easily movable and flexible and became a green-reflecting film to the eye upon an increase in pressure of 0.4 bar. If the pressure was increased again by 0.7 bar, the color turned blue. The process was reversible. The color change of the film occurred in less than 1 s.

### 2. Preparation of a Further Piezochromic Material According to the Present Invention (Including an Appropriately Assembled Film)

### 2.1 Preparation of a Mixture of a Liquid Crystal and a Chirally Optically Active Substance

The optically active substance cholesteryl oleyl carbonate was doped at 27% by weight into the liquid crystal EBBA. The mixture was heated above the clearing point of EBBA to 52° C.

### 2.2 Preparation of a Mixture of a Monofunctional Acrylate Monomer and a Crosslinker

28% by weight of the crosslinker polyethylene glycol diacrylate, M70, was doped into the monomer octadecyl acrylate, and the mixture was heated to 55° C.

The mixtures prepared according to 2.1 and 2.2 were mixed in a ratio of 1:1.9 at a temperature between 45° C. and 50° C. The polymerization initiator used was 0.45% LOCERIN™ (BASF). The piezochromic material was assembled with a layer thickness of 30 µm between two films available under the trademark ZEONORFILM™, each 12 µm thick. Both ZEONORFILM™ films were provided with a maleic anhydride-styrene copolymer orientation layer (< 1 µm). The system was polymerized in UV light for 20 min. Thereafter, the ZEONORFILM™ films could then be easily removed. This yielded a 30 µm thick piezochromic layer. It changed its color from red to blue in less than 1 s upon an increase in pressure of 0.8 bar.

1. A medical device for visually displaying an injection pressure of a fluid, the medical device comprising a fluid path for the fluid and a piezochromic material which exhibits a piezochromic color change when exposed to a pressure of < 100 bar.

2. The medical device according to claim 1, wherein the piezochromic material exhibits the piezochromic color change when exposed to a pressure from 1 bar to 3 bar.

3. The medical device according to claim 1, wherein the piezochromic material exhibits the piezochromic color change within a period of < 1 s.

4. The medical device according to claim 1, wherein the piezochromic material comprises a matrix composed of at least one polymer and/or copolymer, at least one liquid crystal and at least one optically active substance, wherein the optically active substance is distributed in the matrix.

5. The medical device according to claim 4, wherein the at least one liquid crystal is selected from the group consisting of N-(p-ethoxybenzylidene)-p-n-butylaniline, N-(p-methoxybenzylidene)-p-n-butylaniline, 4-alkylphenyl 4-N-alkylbenzoate, cholesteryl benzoate, cholesterol, tolan, alkanoic acids, stilbene, azobenzene, 4-phenylcinnamic acid, p-terphenyl, 1,2-bisbenzoethylene, mixtures of alkylcyanobiphenyls and alkoxycyanobiphenyls, and mixtures of at least two of the aforementioned liquid crystals.

6. The medical device according to claim 4, wherein the at least one optically active substance is selected from the group consisting of 2-octyl 4-[[4-(hexoyl)benzoyl]oxy]benzoate, cholesteryl derivatives and mixtures thereof.

7. The medical device according to claim 4, wherein the polymer and/or copolymer is a poly(meth)acrylate or a copoly(meth)acrylate that has been synthesized from at least one monofunctional monomer and/or at least one multifunctional crosslinker.

8. The medical device according to claim 7, wherein the monomer is selected from the group consisting of benzyl (meth)acrylate, 2-ethylhexyl (meth)acrylate, methoxyethyl (meth)acrylate, octadecyl (meth)acrylate and mixtures of at least two of the aforementioned monomers and/or the crosslinker is selected from the group consisting of 1,4-butanediol di(meth)acrylate, polyethylene glycol diacrylate, polyethylene glycol di(meth)acrylate, 1,10-decanediol di(meth)acrylate, bisphenol A glycerolate (1 glycerol/phenol) diacrylate and mixtures of at least two of the aforementioned crosslinkers.

9. The medical device according to claim 1, wherein the piezochromic material is contained in a housing wall of the medical device that surrounds the fluid path.

10. The medical device according to claim 9, wherein the housing wall comprises at least one multilayer wall section, wherein an intermediate layer of the at least one multilayer wall section comprises the piezochromic material or consists of the piezochromic material.

11. The medical device according to claim 10, wherein the at least one multilayer wall section also comprises an inner layer which covers the intermediate layer on a side facing the fluid path and which comprises a biocompatible material or consists of the biocompatible material.

12. The medical device according to claim 11, wherein the biocompatible material is selected from the group consisting of polyethylene terephthalate, polyetheretherketone, polypropylene, polyphenylsulfone, polyoxymethylene and mixtures of at least two of the aforementioned biocompatible materials.

13. The medical device according to claim 10, wherein the at least one multilayer wall section also comprises an outer layer which covers the intermediate layer on a side facing away from the fluid path and which comprises a transparent material or consists of the transparent material.

14. The medical device according to claim 13, wherein the transparent material is selected from the group consisting of polycarbonate, polymethyl methacrylate, polyurethane and mixtures of at least two of the aforementioned transparent materials.

15. The medical device according to claim 9, wherein the housing wall is a wall of a syringe body of the medical device.

16. The medical device according to claim 9, wherein the housing wall is a lateral wall of a syringe body of the medical device.

17. The medical device according to claim 9, wherein the housing wall is an end wall of a syringe body of the medical device.

18. The medical device according to claim 9, wherein the housing wall is a wall of an outlet nozzle of a syringe body of the medical device.

19. The medical device according to claim 18, wherein the outlet nozzle comprises a branch having a closed end, wherein the piezochromic material is contained in the closed end.

20. The medical device according to claim 1, wherein the piezochromic material is contained in a stopper of a syringe plunger of the medical device.
